(19) United States
(12) Patent Application Publication
(10) Pub. No.: US 2017/0319478 A1
(43) Pub. Date: Nov. 9, 2017

(54) Excipient Suitable for Preparing Creams

(71) Applicant: Johan VAN DAELE, Elversele (BE)

(72) Inventor: Johan VAN DAELE, Elversele (BE)

(21) Appl. No.: 15/525,215

(22) PCT Filed: Nov. 5, 2015
(86) PCT No.: PCT/EP2015/075865

§ 371 Date: May 8, 2017

(30) Foreign Application Priority Data
Nov. 6, 2014 (BE) 2014/0803

(51) Int. Cl. A61K 9/06; A61K 8/02; A61K 47/14; A61K 47/12; A61K 47/10; A61K 47/06; A61K 36/752; A61K 36/534; A61K 36/53; A61K 33/30; A61K 31/197; A61K 9/107; A61K 8/92; A61K 8/67; A61K 8/49; A61K 8/37; A61K 8/36; A61K 8/34; A61K 8/31; A61K 8/27; A61K 8/06; A61K 47/26; A61K 47/44

(52) U.S. Cl. CPC A61K 9/06; A61K 47/44; A61K 8/0216; A61K 47/14; A61K 47/12; A61K 47/10; A61K 47/06; A61K 36/752; A61K 36/534; A61K 36/53; A61K 33/30; A61K 31/197; A61K 9/107; A61K 8/925; A61K 8/922; A61K 8/673; A61K 8/4973; A61K 8/37; A61K 8/361; A61K 8/345; A61K 8/31; A61K 8/27; A61K 8/064; A61K 47/26

(57) ABSTRACT

An excipient, suitable for the preparation of pharmaceutical and/or cosmetic creams comprises an aqueous phase and organic phase comprising organic components. The components comprise natural or synthetic oil, one or more waxes, one or more organic esters. Each of the general formula R1 COO R2, wherein R1 and R2 are both, independently of one another, is a linear or branched alkyl group having 15 to 30 carbon atoms and sorbitan esters.

### FIELD OF THE INVENTION

The present invention relates to an excipient which is usable as a carrier for pharmaceutical and/or cosmetic cream per se, and to pharmaceutical and/or cosmetic creams, which creams can be applied to human or animal skin. The present invention also relates to methods for producing such a cream or excipient.

### BACKGROUND OF THE INVENTION

In the pharmaceutical and cosmetics industries, creams are often produced for application to human or animal tissue, often human or animal skin, in order to obtain a medical or cosmetic effect. The starting point for such creams is often an excipient, to which one or more active constituents or components are added, which active components provide the medical or cosmetic effect. The excipient used is, as it were, the carrier of the active components in the creams.

Currently, it is common to use excipients which are based on an oil-in-water emulsion, wherein the external phase is aqueous and the internal emulsified phase is oil. Often, such an oil-in-water emulsion is insufficiently moisturizing and replenishing for dry and damaged skin, and fatty active constituents are not always equally well delivered to the skin because of emulsified.

An alternative is an excipient which comprises exclusively fat or oil. Oils of this type have the drawback of closing up the skin, with the result that the skin is no longer able to get rid of its waste substances, which can often cause inflammation and/or slower recovery. In addition, excipients of this type are considered unpleasant to use and do not penetrate well into the skin.

Attempts have been made to develop a water-in-oil emulsion, in which water is the internal phase and oil is the external phase. Such an excipient could be used as a cream on its own or as an excipient for creams to counteract dry and therefore often itchy skin, or as wound-healing cream. Such excipients have often proven relatively unstable and/or to have little cooling effect, the latter being desirable in order to reduce itching.

### SUMMARY OF THE INVENTION

It is an object of the invention to provide an excipient or cream which is a water-in-oil emulsion and which is suitable for use as an excipient or cream per se for pharmaceutical and/or cosmetic purposes. The excipient according to the present invention has the advantage of remaining stable for a prolonged period of time when stored at room temperature with little or even no separation into an aqueous and an organic phase. Another object of the invention is to provide a method for producing an excipient according to the present invention.

Another object of the invention is to develop a water-in-oil cream (also referred to as a w/o cream) which can be combined with a wide range of active constituents, is as hypo-allergenic as possible, and is as low-cost as possible without any detriment to quality.

One or more of the above objects and/or advantages is achieved by the excipient according to the present invention.

One or more of the abovementioned objects and/or advantages is also achieved by the methods according to the present invention.

A first aspect of the present invention provides an excipient which is suitable for preparing pharmaceutical and/or cosmetic creams. The excipient comprises:
10 to 73% by weight of aqueous phase which comprises the following components
   0.01% by weight to 2.25% by weight of preservative;
   97.75% by weight to 99.99% by weight of purified water;
organic phase comprising the organic components
   natural and/or synthetic oils and/or fats;
   1% by weight to 15% by weight of one or more waxes; 1% by weight to 15% by weight of one or more organic esters, each of the general formula R1-COO—R2, where R1 and R2 are both, independently of one another, a linear or branched alkyl group having 11 to 30 carbon atoms;
   1% by weight to 8% by weight of sorbitan esters;
      which percentages by weight of the components of the aqueous phase are expressed as the weight of the component over the total weight of the aqueous phase, the percentages by weight of the components of the organic phase are expressed as the weight of the component over the total weight of the excipient, and the percentage by weight of the aqueous phase is expressed as the weight of the aqueous phase over the total weight of the excipient.

According to some embodiments, the organic components may comprise natural and/or synthetic oils.

According to some embodiments, the organic components may comprise 5% by weight to 15% by weight of one or more organic esters, each having the general formula R1-COO—R2, wherein R1 and R2 are both, independently of one another, a linear or branched alkyl group having 11 to 30 carbon atoms.

According to some embodiments, the excipient may be a water-in-oil emulsion.

The term “water-in-oil” is also written as w/o.

According to some embodiments, the organic phase may comprise 20 to 70% by weight of natural or synthetic oils, preferably selected from the group consisting of paraffin oil, vaseline, preferably white vaseline, and combinations of the two.

Unless specifically stated otherwise, all percentages by weight (% by weight) of a component are based on the total weight of the product in which the component is contained.

The organic esters perform a function of auxiliary emulsifier and viscosity regulator whereas the sorbitan esters are used essentially as emulsifier.

An excipient is a constituent of a pharmaceutical or cosmetic preparation which dissolves or suspends the other components (for example active components which provide a medical or cosmetic effect if the preparation is applied to tissue, for example skin, of a human or animal) and provides the desired consistency of the preparation. The excipient is what is known as the carrier of the cream, and the cream is based on the excipient.

Preferably, the aqueous phase comprises 98% by weight to 99.5% by weight of purified water, which percentage by weight of purified water is expressed as the weight of the component over the total weight of the aqueous phase.

The preservative may comprise one or more components which have a preserving property.

The preservative may be a combination of
0.5% by weight to 2% by weight of propylene glycol;
0.05% by weight to 0.15% by weight of methyl parahydroxybenzoate;
0.01% by weight to 0.1% by weight of propyl parahydroxybenzoate,
   which percentages by weight of the components of the aqueous phase are expressed as the weight of the component over the total weight of the aqueous phase.

According to some embodiments, the natural or synthetic oil may comprise paraffin oil.

According to some embodiments, the natural or synthetic oil may comprise paraffin oil and vaseline, in which case the weight ratio of paraffin oil to vaseline is greater than 1.

Preferably, the weight ratio of paraffin oil to vaseline is more than 50/50, such as more than 2.5, for example in the range from 80/20 to 99.99/0.01. The use of vaseline as part of the natural or synthetic oils has the advantage that the stability of the excipient, or of the cream based on the excipient, can be made less dependent on the ambient temperature at which the excipient or the cream is stored. The addition of vaseline gives a product which will remain stable for longer in the case of excipients or creams which are stored at higher ambient temperatures, for example in tropical regions.

The term “liquid paraffin” means Paraffinum liquidum Ph. Eur. having a viscosity of between 110 mPa·s and 230 mPa·s determined by method 2.2.9 described in Ph. Eur. 7.0.

Ph. Eur. 7.0 means “the European Pharmacopoeia, version 7.0.”

According to some embodiments, the excipient may comprise more than 15% by weight, for example for more than 20% by weight of aqueous phase.

Preferably, the excipient comprises more than 25% by weight, such as 25% by weight or even more than 30% by weight of aqueous phase.

The addition of more water causes a greater cooling effect when the excipient or a cream based on the excipient is applied to the skin of a human or animal body.

According to some embodiments, the natural or synthetic oil may be present in a concentration of between 53% by weight and 63% by weight.

In particular, the liquid paraffin can provide a good consistency of the cream, and moreover is hypo-allergenic, making it accessible for wide public use. In addition, it forms a base in which lipophilic molecules can be dissolved.

Organic esters are esters in which R1 and R2, independently of one another, contain 11 to 30, preferably 15 to 24 carbon atoms.

Excipients according to the invention are formed in such a way that the excipient forms a water-in-oil base with an optimum spreadability and stability, on the one hand, and which, on the other hand, breaks down when spread, so as to have a cooling effect.

According to some embodiments, the natural or synthetic oils have a viscosity of between 110 mPa·s and 230 mPa·s, which viscosity is determined by method 2.2.9 described in Ph. Eur. 7.0.

According to some embodiments, wax may be present in an amount from 5.5% to 9.5% by weight.

Wax in particular provides a good consistency of the excipient.

According to some embodiments, the wax used may be cera alba.

According to some embodiments, the one or more organic esters may be present in the excipient in an amount of from 7% by weight to 14% by weight.

According to some embodiments, the one or more organic esters may comprise or be cetyl palmitate.

According to some embodiments, the excipient may comprise:
in the aqueous phase the components
   16 to 20% by weight of purified water
   0.8 to 2% by weight of propylene glycol, which percentage by weight is expressed as the weight of the component over the total weight of the aqueous phase;
   0.07 to 0.09% by weight of methyl parahydroxybenzoate, which percentage by weight is expressed as the weight of the component over the total weight of the aqueous phase
   less than 0.03% by weight of propyl parahydroxybenzoate, which percentage by weight is expressed as the weight of the component over the total weight of the aqueous phase
in the organic phase the organic components
   53 to 63% by weight of paraffin oil
   7 to 9% by weight of cera alba
   7 to 12% by weight of cetyl palmitate
   2 to 4% by weight of sorbitan sesquioleate
      the percentages by weight of the components in the organic and aqueous phase are expressed, unless specified otherwise, as the weight of the component over the total weight of the excipient.

According to some embodiments, the excipient may comprise more than 25% by weight of purified water.

According to some embodiments, the excipient may comprise:
in the aqueous phase the components
   35 to 41% by weight of purified water
   0.3 to 2% by weight of propylene glycol, which percentage by weight is expressed as the weight of the component over the total weight of the aqueous phase
   0.02 to 0.04% by weight of methyl parahydroxybenzoate, which percentage by weight is expressed as the weight of the component over the total weight of the aqueous phase
   less than 0.01% by weight of propyl parahydroxybenzoate, which percentage by weight is expressed as the weight of the component over the total weight of the aqueous phase
in the organic phase the organic components
   35 to 50% by weight of paraffin oil
   3 to 8% by weight of cera alba
   4 to 9% by weight of cetyl palmitate
   3.5 to 6% by weight of sorbitan sesquioleate
      the percentages by weight of the components in the organic and aqueous phase are expressed, unless specified otherwise, as the weight of the component over the total weight of the excipient.

According to some embodiments, the excipient may comprise:
in the aqueous phase the components
   35 to 41% by weight of purified water
   0.3 to 2% by weight of propylene glycol, which percentage by weight is expressed as the weight of the component over the total weight of the aqueous phase
   0.02 to 0.04% by weight of methyl parahydroxybenzoate, which percentage by weight is expressed as the weight of the component over the total weight of the aqueous phase
   less than 0.01% by weight of propyl parahydroxybenzoate, which percentage by weight is expressed as the weight of the component over the total weight of the aqueous phase
in the organic phase the organic components
   35 to 50% by weight of paraffin oil and vaseline wherein the weight ratio of paraffin oil to vaseline varies and is greater than 1;
   3 to 8% by weight of cera alba
   4 to 9% by weight of cetyl palmitate
   3.5 to 6% by weight of sorbitan sesquioleate
      the percentages by weight of the component in the organic and aqueous phase are expressed, unless specified otherwise, as the weight of the component over the total weight of the excipient.

The excipient according to the invention serves as a carrier for a cosmetic or medicinal cream, or can either be used as such a cream, for example for the treatment of dry skin, itching, wounds, burns, burns caused by radiotherapy, ichthyosis, dry eczema, bedsores (decubitus), psoriasis, scars, pigment disorders and many other dermatological diseases and skin disorders. The excipient used as a cream can have a cooling, anti-itching, pain-relieving, wound-healing, skin-repairing and nutritive effect on, for example, burns or for example burns caused by irradiation, on dry skin symptoms such as dry skin, dry eczema and ichthyosis, on bedsores, psoriasis, and many other dermatological diseases and skin disorders.

A second aspect of the present invention provides a method for producing an excipient, which method comprises the steps of:
providing aqueous components comprising purified water and a preservative;
providing organic components comprising
   natural and/or synthetic oils and/or fats;
   one or more waxes;
   one or more organic esters, each of the general formula R1-COO—R2,
   wherein R1 and R2 are both, independently of one another, a linear or branched alkyl group having 11 to 30 carbon atoms;
   one or more sorbitan esters;
preparing a water-in-oil emulsion by
   combining the aqueous and organic components
   heating to above 60° C.,
   emulsifying, and
   cooling to less than 45° C.;
leaving the emulsion to stand for a period of longer than or equal to 2 hours; thoroughly mixing the emulsion obtained, so that the excipient is obtained.

According to some embodiments, the organic components may comprise natural and/or synthetic oils.

According to some embodiments, the organic components may comprise 1% by weight to 15% by weight, for example 5% by weight to 15% by weight, of one or more organic esters, each having the general formula R1-COO—R2, wherein R1 and R2 are both, independently of one another, a linear or branched alkyl group having 11 to 30 carbon atoms.

According to some embodiments, said leaving to stand may be such that no action is taken during said leaving to stand, e.g. no heating or no other action.

According to some embodiments, the thorough mixing may take place at a reduced pressure of less than or equal to 0.7. bara.

Bara means the absolute pressure, expressed in bar, where 1 bar is equal to 100 000 Pascal. 0 bara therefore means absolute vacuum.

According to some embodiments, during the preparation of a water-in-oil emulsion, first of all a mixture of the aqueous and the organic components may be provided, which mixture of the components is emulsified.

According to some embodiments, during the preparation of the emulsion, the aqueous and organic components, if appropriate provided as a mixture, can be heated to a temperature of between 75° C. and 80° C.

According to some embodiments, the heating may take place with gentle stirring.

The term gentle stirring is to be understood as meaning the stirring of the components, or if appropriate the mixture, preferably with the aid of a mixer, at a speed of less than 1500 rpm.

According to some embodiments, the mixture, after said heating, can be cooled back down until a temperature of below 40° C. is reached.

According to some embodiments, the cooling can take place with gentle stirring.

According to some embodiments, the cooling can take place with the aid of forced cooling.

Forced cooling means that thermal energy is removed from the product, in this case the emulsion, by means of a cooling fluid which is at a temperature below the ambient temperature, such as cooling water.

According to some embodiments, during the cooling the temperature of the emulsion may drop at a rate of from 0.5 to 1.5° C. per minute.

According to some embodiments, during the cooling the emulsion can be scraped off the wall of the mixer.

According to some embodiments, the mixture, after said heating, can be emulsified in a mixer at a speed of more than 1500 rpm.

It is preferable to use a Stephan® mixer which is set at a speed of from 1500 to 4000 rpm in order to emulsify the components in the mixture.

The use of the Stephan® mixer at 1500 to 4000 rpm delivers high shear mixing. It is preferable to use the Stephan® mixer at a speed of around 3000 rpm, for example 3000 rpm. In this way, it is possible to create sufficient small spheres of water in the water-in-oil emulsion.

According to some embodiments, the emulsifying can take place over a period of from 2 to 10 minutes.

According to some embodiments, said steps of heating, emulsifying and cooling in the preparation of a water-in-oil emulsion can take place under a vacuum of less than or equal to 0.7 bara.

According to some embodiments of the invention, said heating and cooling can take place under a vacuum of less than 0.7 bara. It is preferable to apply pressure of less than 0.5 bara, or even less than 0.3 bara.

According to some embodiments, after said cooling, the emulsion can be left to stand for a period of at least 4 hours, after which the emulsion is stirred through again.

The standing time is preferably more than 6 hours. During the standing time, the temperature can preferably further be brought to ambient temperature, for example to 20 to 25° C.

According to some embodiments, said leaving to stand may be such that no action is taken during said leaving to stand, e.g. no heating or no other action.

According to some embodiments, the thorough mixing may take place over a period of from 0.5 to 10 minutes, for example over a period of from 2 to 10 minutes.

According to some embodiments, the components are provided in a weight ratio as described for the excipients according to the first aspect of the invention.

According to some embodiments, the organic and aqueous components may comprise 10 to 73% by weight of aqueous components, which aqueous components comprise the following components:
0.01% by weight to 2.25% by weight of preservative;
97.75% by weight to 99.99% by weight of purified water;
and wherein the organic components comprise the following components:
natural and/or synthetic oils and/or fats;
1% by weight to 15% by weight of one or more waxes;
1% by weight to 15% by weight of one or more organic esters, each of the general formula R1-COO—R2, where R1 and R2 are both, independently of one another, a linear or branched alkyl group having 11 to 30 carbon atoms;
1% by weight to 8% by weight of sorbitan esters;
   which percentages by weight of the aqueous components are expressed as the weight of the component over the total weight of the aqueous components, the percentages by weight of the organic components are expressed as the weight of the component over the total weight of the organic and aqueous components, and the percentage by weight of the aqueous components together is expressed as the weight of the aqueous phase over the total weight of the organic and aqueous components.

According to some embodiments,
the aqueous components may comprise
   16 to 20% by weight of purified water
   0.8 to 2% by weight of propylene glycol, which percentage by weight is expressed as the weight of the component over the total weight of the aqueous phase
   0.07 to 0.09% by weight of methyl parahydroxybenzoate, which percentage by weight is expressed as the weight of the component over the total weight of the aqueous phase
   less than 0.03% by weight of propyl parahydroxybenzoate, which percentage by weight is expressed as the weight of the component over the total weight of the aqueous phase
the organic components may comprise
   53 to 63% by weight of paraffin oil
   7 to 9% by weight of cera alba
   7 to 12% by weight of cetyl palmitate
   2 to 4% by weight of sorbitan sesquioleate
      the percentages by weight of the components in the organic and aqueous phase are expressed, unless specified otherwise, as the weight of the component over the total weight of the excipient.

According to some embodiments, the organic and aqueous components may comprise more than 15 or even more than 20% by weight of aqueous components.

According to some embodiments:
the aqueous components may comprise:
   35 to 41% by weight of purified water
   0.3 to 2% by weight of propylene glycol, which percentage by weight is expressed as the weight of the component over the total weight of the aqueous phase
   0.02 to 0.04% by weight of methyl parahydroxybenzoate, which percentage by weight is expressed as the weight of the component over the total weight of the aqueous phase
   less than 0.01% by weight of propyl parahydroxybenzoate, which percentage by weight is expressed as the weight of the component over the total weight of the aqueous phase
the organic components may comprise
   35 to 50% by weight of paraffin oil
   3 to 8% by weight of cera alba
   4 to 9% by weight of cetyl palmitate
   3.5 to 6% by weight of sorbitan sesquioleate
      the percentages by weight of the components are expressed, unless specified otherwise, as the weight of the component over the total weight of the aqueous and organic components.

According to some embodiments:
the aqueous components may comprise
   35 to 41% by weight of purified water
   0.3 to 2% by weight of propylene glycol, which percentage by weight is expressed as the weight of the component over the total weight of the aqueous phase
   0.02 to 0.04% by weight of methyl parahydroxybenzoate, which percentage by weight is expressed as the weight of the component over the total weight of the aqueous phase
   less than 0.01% by weight of propyl parahydroxybenzoate, which percentage by weight is expressed as the weight of the component over the total weight of the aqueous phase
the organic components may comprise
   35 to 50% by weight of paraffin oil and vaseline wherein the weight ratio of paraffin oil to vaseline varies and is greater than 1;
   3 to 8% by weight of cera alba
   4 to 9% by weight of cetyl palmitate
   3.5 to 6% by weight of sorbitan sesquioleate
      the percentages by weight of the components are expressed, unless specified otherwise, as the weight of the component over the total weight of the aqueous and organic components.

According to a third aspect of the invention, an excipient is obtained by using one of the methods in accordance with the second aspect of the invention.

According to some embodiments of the invention, after the standing time and before the mixing again, active components can be added, in order to obtain a cosmetic or medicinal cream.

The methods according to the present invention have the advantage of producing a stable excipient with no or only minimal separation back into an aqueous and an organic phase during storage.

The excipient according to the invention may form a pharmaceutical carrier for the processing of one or more active ingredients which give the end product a pharmaceutical or medicinal action.

When the excipient according to the invention is rubbed onto the skin, a cooling effect can occur as a result of the emulsion breaking open and/or the water evaporating out of the cream.

The excipient according to the invention, used as a carrier for a pharmaceutical or cosmetic cream, or as the cream itself, is found to be notably pleasant to use as a result of the cooling effect and good penetration through the skin barrier in the case of dry and/or dehydrated skin, yet a protective but not occlusive film still remains behind, ensuring a prolonged nutritive and therapeutic effect.

A third aspect of the invention provides a cosmetic or medicinal cream. The term “cream” is used to denote a preparation which is a mixture of lipid phase and aqueous phase, stabilized with an emulsifier. A cream preferably has an optimum viscosity. It is possible for a component to be worked into the cream, either as a solution or as a suspension.

A fourth aspect of the invention provides a cosmetic or medicinal cream which comprises 75% by weight to 100% by weight of excipient according to the first aspect of the invention.

According to some embodiments of the invention, this cosmetic or medicinal cream may comprise 75% by weight to 100% by weight of excipient, which excipient has been obtained by one of the methods according to the second aspect of the invention.

According to some embodiments, the one or more active components may comprise one or more components selected from the group consisting of calcium pantothenate, rosemary essential oil, lavender spike essential oil, lemon essential oil and peppermint essential oil.

According to some embodiments, the one or more active components may comprise calcium pantothenate.

According to some embodiments, the one or more active components may comprise lavender essential oil.

According to some embodiments, the one or more active components may comprise lavender essential oil and rosemary essential oil.

According to some embodiments, the one or more active components may comprise lavender essential oil, rosemary essential oil and lemon essential oil.

According to some embodiments, the one or more active components may comprise lavender essential oil, rosemary essential oil, lemon essential oil and peppermint essential oil.

According to some embodiments, the one or more active components may comprise lavender spike essential oil.

According to some embodiments, the one or more active components may comprise essential oil of Rosmarinus officinalis var. cineole.

Calcium pantothenate and rosemary essential oil are added for their tissue-restoring effect. It is preferable to use essential oil of Rosmarinus officinalis var. cineole, although var. camphor and/or verbenone or combinations of these variations are possible. The rosemary essential oil also has an anti-infectious action. Lavender spike (Lavendula spica) essential oil has a wound-healing effect, while lemon essential oil (produced from the peel) and peppermint essential oil both further reinforce the refreshing effect of the excipient.

According to some embodiments, the cream may comprise 75% by weight to 99.9% by weight of excipients, and 0.1% by weight to 25% by weight of one or more active components, the percentages by weight of the components in the cosmetic or medicinal cream being expressed as the weight of the component over the total weight of the cosmetic or medicinal cream.

According to some embodiments, the one or more active components may comprise one or more components selected from the group consisting of calcium pantothenate, rosemary essential oil, lavender spike essential oil, lemon essential oil and peppermint essential oil.

According to some embodiments, the one or more active components may comprise essential oil of Rosmarinus officinalis var. cineole.

According to some embodiments, the one or more active components may comprise one or more zinc-containing compounds.

According to some embodiments, the one or more zinc-containing compounds may be zinc-containing compounds selected from the group consisting of zinc oxide, zinc citrate and zinc sulphate.

According to some embodiments, the one or more active zinc-containing compounds may form 0.1% by weight to 10% by weight of the cosmetic or medicinal cream, which percentage by weight is expressed as the total weight of the zinc-containing compounds with respect to the weight of the cosmetic or medicinal cream.

Zinc-containing compounds may be encountered in various wound-healing, enzymatic systems, and are suitable for use in wound-healing cosmetic or medicinal creams, for example when treating bedsores.

A fifth aspect of the invention provides a method for producing a cosmetic or medicinal cream, which cosmetic or medicinal cream comprises an excipient and active compounds. The excipient is produced by one of the methods according to the second aspect of the invention. The one or more active components are added to the excipient and then stirred into the excipient.

A sixth aspect of the invention provides a method for producing a cosmetic or medicinal cream, which cosmetic or medicinal cream comprises an excipient and one or more active components. The excipient is produced by one of the methods according to the second aspect of the invention. During the production of the excipient, one or more active components are added.

According to some embodiments, after said leaving to stand and before said thorough mixing, one or more active components can be added.

According to some embodiments, one or some of the plurality of active components are added as early as during the production of the excipient, and others of the plurality of active components are added after the said standing time and before the said thorough mixing. Preferably, calcium pantothenate is added as early as during the production of the excipient, more specifically at the beginning of the preparation of the excipient.

According to some embodiments, one or more active components, the aqueous and organic components can be combined before the preparation of the water-in-oil emulsion.

According to some embodiments, the one or more active components may comprise one or more components selected from the group consisting of calcium pantothenate, rosemary essential oil, lavender spike essential oil, lemon essential oil and peppermint essential oil.

According to some embodiments, the one or more active components may comprise calcium pantothenate.

According to some embodiments, the one or more active components may comprise lavender essential oil.

According to some embodiments, the one or more active components may comprise lavender essential oil and rosemary essential oil.

According to some embodiments, the one or more active components may comprise lavender essential oil, rosemary essential oil and lemon essential oil.

According to some embodiments, the one or more active components may comprise lavender essential oil, rosemary essential oil, lemon essential oil and peppermint essential oil.

According to some embodiments, the one or more active components may comprise lavender spike essential oil.

According to some embodiments, the one or more active components may comprise essential oil of Rosmarinus officinalis var. cineole.

According to some embodiments, the one or more active components may comprise one or more zinc-containing compounds.

According to some embodiments, the one or more zinc-containing compounds may be zinc-containing compounds selected from the group consisting of zinc oxide, zinc citrate and zinc sulphate.

According to some embodiments, the one or more active zinc-containing compounds may form from 0.1% by weight to 10% by weight of the cosmetic or medicinal cream, which percentage by weight is expressed as the total weight of the zinc-containing compounds with respect to the weight of the cosmetic or medicinal cream.

A seventh aspect of the invention provides cosmetic or medicinal cream, which excipient was obtained by one of the methods according to the sixth aspect of the invention.

The one or more active components in the cosmetic or medicinal cream may, inter alia, be: essential oils, natural moisturizing factors, components for stimulating the generation of skin cells or for promoting the restoration of skin cells, and the like.

The excipients according to the present invention, and those obtained by the method according to the present invention, and creams based on these excipients, are therefore extremely suitable for the treatment of burns or for providing a cooling effect after the application of a tattoo to human or animal skin.

Moreover, the excipients according to the present invention, and those obtained by the method according to the present invention, and creams based on these excipients, can have a preventive use for preventing or reducing the risk of epidermolysis, erythema, irritation, pain or dryness of the skin, for example caused by radiotherapy. The excipients according to the present invention, and those obtained by the method according to the present invention, and creams based on these excipients, can also have a curative use for epidermolysis, erythema, irritation, burns, pain or dryness of the skin, for example caused by the application of tattoos, radiotherapy, bedsores, eczema, psoriasis, keratosis pilaris and similar disorders, or also as a lubricant for sexual contact, for example during sexual intercourse.

The above and other features, properties and advantages of the present invention will be explained with the aid of the following examples of embodiments. The description of these examples of embodiments is given by way of explanation, without being intended to restrict the scope of the invention.

### DESCRIPTION OF EXAMPLES OF EMBODIMENTS

The present invention is described below making use of specific embodiments.

It should be noted that the term “comprising” as used for example in the claims is not to be interpreted in any limiting sense implying any restriction to the following elements, features and/or steps. The term “comprising” does not exclude the presence of other elements, features or steps. Thus, the scope of an expression “an object comprising the elements A and B” is not limited to an object which includes only the elements A and B. The scope of an expression “a method comprising steps A and B” is not limited to a method which includes only steps A and B.

In the context of the present invention, these expressions mean only that the relevant elements or steps constitute the elements or steps A and B for the purposes of the invention.

In the specification which follows, reference is made to “an embodiment” or “the embodiment”. Such reference means that a specific element or feature, described on the basis of this embodiment, is included in at least this one embodiment. The occurrence of the term “in an embodiment” or “in one embodiment” at various places in this description, however, does not necessarily refer to the same embodiment, although it may refer to the same embodiment.

Furthermore, the properties or the features may be combined in any suitable way in one or more embodiments, as will be clear to a person skilled in the art.

An embodiment of an excipient according to the invention has a composition as follows:

| | | |
|---|---|---|
| Liquid paraffin/paraffinum liquidum | 60.51 | g |
| White beeswax/cera alba | 7.68 | g |
| Cetyl palmitate | 9.60 | g |
| Sorbitan sesquioleate/arlacel 83 | 3.00 | g |
| Aqua conservans | 19.21 | g |
| Total weight | 100 | g |

Cetyl palmitate is an organic ester having the general formula R1-COO—R2, wherein R1 is a linear alkyl group having 15 carbon atoms and R2 is a linear alkyl group having 16 carbon atoms.

The components liquid paraffin, white beeswax, cetyl palmitate and sorbitan sesquioleate together form the organic phase, which forms 80.79% by weight of the excipient. The aqueous phase, referred to above as aqua conservans, forms the remaining 19.21% by weight. This aqueous phase or aqua conservans consists of:

| | |
|---|---|
| Purified water | 99.00 g |
| Propylene glycol | 0.90 g |
| Methyl parahydroxybenzoate | 0.08 g |
| Propyl parahydroxybenzoate | 0.02 g |
| Total | 100.00 g |

The excipient is formed by combining the ingredients in a suitable container, provided with a mixer, preferably a Stephan mixer and suitable for evacuation.

After the air has been evacuated from the container to a vacuum of around 0.25 bara, the ingredients are gently stirred using the Stephan® mixer while the temperature is increased to 80° Celsius. This is followed by high-shear mixing of the mixture for 3 minutes using the Stephan mixer, mixing at 3000 rpm. Thereafter, the emulsion is actively cooled to below 40° Celsius, if appropriate to room temperature. The vacuum is removed.

The excipient obtained in this way is then left to stand for a few hours, at least 2 but preferably 6 hours, and preferably approximately 24 hours.

Then, if appropriate the active components are added, and the excipient (together with any active components) again undergoes high-shear mixing in the Stephan mixer, mixing at 3000 rpm for a few minutes. This mixing may if appropriate takes place after the air has been evacuated from the container to a vacuum of around 0.25 bara.

This produces the excipient according to the invention.

The excipient is also mixed again if no further active components are added in order to obtain a cosmetic or medicinal cream. This is in order to obtain a homogeneous and stable cream or excipient with few or no lumps.

This excipient can be used, inter alia, for the production of an itch-reducing and/or burn/wound-repairing cream. The following different active components are added to the excipient in order to obtain a cream, i.e. a w/o emulsion, having the following composition:

| | | |
|---|---|---|
| Calcium pantothenate | 1.00 | g |
| Rosemary essential oil | 0.50 | g |
| Lavender spike essential oil | 0.09 | g |
| Lemon essential oil (peel) | 0.37 | g |
| Peppermint essential oil | 0.37 | g |
| Liquid paraffin/paraffinum liquidum | 58.58 | g |
| White beeswax/cera alba | 7.61 | g |
| Cetyl palmitate | 9.51 | g |
| Sorbitan sesquioleate/arlacel 83 | 2.97 | g |
| Aqua conservans | 19.01 | g |
| Total weight | 100 | g |

The active components as listed above are added and mixed with the excipient.

Calcium pantothenate and rosemary essential oil are added for their tissue-restoring effect. It is preferable to use essential oil of Rosmarinus officinalis var. cineole, although var. camphor and/or verbenone or combinations of these variations are possible.

The rosemary essential oil also has an anti-infectious action. Lavender spike (Lavendula spica) essential oil provides a wound-healing effect, while lemon essential oil (produced from the peel) and peppermint essential oil both further increase the refreshing effect of the excipient.

Another embodiment of an excipient according to the invention has a composition as follows:

| | | |
|---|---|---|
| Liquid paraffin/paraffinum liquidum | 44.32 | g |
| White beeswax/cera alba | 5.46 | g |
| Cetyl palmitate | 6.82 | g |
| Sorbitan sesquioleate/arlacel 83 | 4.96 | g |
| Aqua conservans | 38.45 | g |
| Total weight | 100 | g |

The components liquid paraffin, white beeswax, cetyl palmitate and sorbitan sesquioleate together form the organic phase, forming 61.55% by weight of the excipient. The aqueous phase, referred to above as aqua conservans, forms the remaining 38.45% by weight. This aqueous phase or aqua conservans is the same as that used in the first embodiment.

The excipient is formed using the same procedure as described above in connection with the first embodiment.

In a third embodiment, some of the liquid paraffin is replaced by vaseline, preferably white vaseline, in the same quantities by weight (i.e. 1 g of paraffin is replaced by 1 gram of vaseline). For example, half or a quarter of the paraffin can be replaced by vaseline. An excipient containing vaseline can be obtained by a similar or even identical preparation method to that explained in connection with the first and second embodiments.

These excipients can be used for, inter alia, the production of an itching-reducing and/or burn/wound-repairing cream. The following different active components are added to the excipient in order to obtain a cream, i.e. a w/o emulsion, having the following composition:

The same quantity of essential oils are added, either after the excipient has been made or before the thorough mixing of the excipient, or added as an additional ingredient to the aqueous and organic components before the heating and emulsifying of these components.

It will be clear that, although the embodiments and/or the materials have been discussed for the purpose of providing embodiments in accordance with the present invention, various alterations or changes may be made without deviating from the scope and/or the spirit of the present invention.

1-28. (canceled)

29. Excipient, suitable for the preparation of pharmaceutical and/or cosmetic creams, which excipient comprises:
10 to 73% by weight of aqueous phase which comprises the following components:
0.01% by weight to 2.25% by weight of preservative;
97.75% by weight to 99.99% by weight of purified water;
organic phase comprising the organic components
natural and/or synthetic oils and/or fats;
1% by weight to 15% by weight of one or more waxes;
1% by weight to 15% by weight of one or more organic esters, each of the general formula R1-COO—R2, wherein R1 and R2 are both, independently of one another, a linear or branched alkyl group having 11 to 30 carbon atoms;
1% by weight to 8% by weight of sorbitan esters;
which percentages by weight of the components of the aqueous phase are expressed as the weight of the component over the total weight of the aqueous phase, the percentages by weight of the components of the organic phase being expressed as the weight of the component over the total weight of the excipient and the percentage by weight of the aqueous phase being expressed as the weight of the aqueous phase over the total weight of the excipient, wherein the excipient is a water-in-oil emulsion.

30. Excipient according to claim 29, wherein the organic phase comprises 20 to 70% by weight of natural or synthetic oil, preferably selected from the group consisting of paraffin oil, vaseline, preferably white vaseline, and combinations of the two.

31. Excipient according to claim 29, wherein the natural or synthetic oil comprises paraffin oil or comprises paraffin oil and vaseline, wherein the weight ratio of paraffin oil to vaseline is greater than 1.

32. Excipient according to claim 29, comprising more than 15% by weight of aqueous phase.

33. Excipient according to claim 29, wherein the natural or synthetic oil is present in a concentration of between 53% by weight and 63% by weight or wherein the natural or synthetic oil has a viscosity of between 110 mPa·s and 230 mPa·s, which viscosity is determined by the method 2.2.9 described in Ph. Eur 7.0.

34. Excipient according to claim 29, wherein wax is present in an amount of from 5.5% to 9.5% by weight or wherein the wax used is cera alba.

35. Excipient according to claim 29, wherein the one or more organic esters are present in the excipient in an amount of from 7% by weight to 14% by weight or wherein the one or more organic esters comprises cetyl palmitate.

36. Excipient according to claim 29, comprising:
in the aqueous phase the components
16 to 20% by weight of purified water
0.8 to 2% by weight of propylene glycol, which percentage by weight is expressed as the weight of the component over the total weight of the aqueous phase
0.07 to 0.09% by weight of methyl parahydroxybenzoate, which percentage by weight is expressed as the weight of the component over the total weight of the aqueous phase
less than 0.03% by weight of propyl parahydroxybenzoate, which percentage by weight is expressed as the weight of the component over the total weight of the aqueous phase
in the organic phase the organic components
53 to 63% by weight of paraffin oil
7 to 9% by weight of cera alba
7 to 12% by weight of cetyl palmitate
2 to 4% by weight of sorbitan sesquioleate
the percentages by weight of the components in the organic and aqueous phases are expressed, unless specified otherwise, as the weight of the component over the total weight of the excipient.

37. Excipient according to claim 29, comprising more than 25% by weight of purified water.

38. Excipient according to claim 37, comprising:
in the aqueous phase the components
35 to 41% by weight of purified water
0.3 to 2% by weight of propylene glycol, which percentage by weight is expressed as the weight of the component over the total weight of the aqueous phase
0.02 to 0.04% by weight of methyl parahydroxybenzoate, which percentage by weight is expressed as the weight of the component over the total weight of the aqueous phase
less than 0.01% by weight of propyl parahydroxybenzoate, which percentage by weight is expressed as the weight of the component over the total weight of the aqueous phase
in the organic phase the organic components
35 to 50% by weight of paraffin oil
3 to 8% by weight of cera alba
4 to 9% by weight of cetyl palmitate
3.5 to 6% by weight of sorbitan sesquioleate
the percentages by weight of the components in the organic and aqueous phases are expressed as the weight of the component over the total weight of the excipient.

39. Excipient according to claim 37, comprising:
in the aqueous phase the components
35 to 41% by weight of purified water
0.3 to 2% by weight of propylene glycol, which percentage by weight is expressed as the weight of the component over the total weight of the aqueous phase
0.02 to 0.04% by weight of methyl parahydroxybenzoate, which percentage by weight is expressed as the weight of the component over the total weight of the aqueous phase
less than 0.01% by weight of propyl parahydroxybenzoate, which percentage by weight is expressed as the weight of the component over the total weight of the aqueous phase
in the organic phase the organic components
35 to 50% by weight of paraffin oil and vaseline wherein the weight ratio of paraffin oil to vaseline varies and is greater than 1;
3 to 8% by weight of cera alba
4 to 9% by weight of cetyl palmitate
3.5 to 6% by weight of sorbitan sesquioleate
the percentages by weight of the components in the organic and aqueous phases are expressed as the weight of the component over the total weight of the excipient.

40. Cosmetic or medicinal cream comprising 75% by weight to 100% by weight of excipient according to claim 29.

41. Cosmetic or medicinal cream according to claim 40, which cream comprises 75% by weight to 99.9% by weight of excipient, and 0.1% by weight to 25% by weight of one or more active components, the percentages by weight of the components in the cosmetic or medicinal cream being expressed as the weight of the component over the total weight of the cosmetic or medicinal cream.

42. Cosmetic or medicinal cream according to claim 40, wherein the one or more active components comprise one or more components selected from the group consisting of calcium pantothenate, rosemary essential oil, lavender spike essential oil, lemon essential oil and peppermint essential oil and/or wherein the one or more active components comprise calcium pantothenate and/or wherein the one or more active components comprise lavender essential oil or wherein the one or more active components comprise lavender essential oil and rosemary essential oil.

43. Cosmetic or medicinal cream according to claim 40, wherein the one or more active components comprise lavender essential oil, rosemary essential oil and lemon essential oil.

44. Cosmetic or medicinal cream according to claim 40, wherein the one or more active components comprise lavender essential oil, rosemary essential oil, lemon essential oil and peppermint essential oil.

45. Cosmetic or medicinal cream according to claim 40, wherein the one or more active components comprise lavender spike essential oil and/or wherein the one or more active components comprise essential oil of Rosmarinus officinalis var cineole.

46. Cosmetic or medicinal cream according to claim 40, wherein the one or more active components comprise one or more zinc-containing compounds.

47. Cosmetic or medicinal cream according to claim 46, wherein one or more zinc-containing compounds are zinc-containing compounds selected from the group consisting of zinc oxide, zinc citrate and zinc sulphate.

48. Cosmetic or medicinal cream according to claim 46, wherein the one or more active zinc-containing compounds form 0.1% by weight to 10% by weight of the cosmetic or medicinal cream, which percentage by weight is expressed as the total weight of the zinc-containing compounds with respect to the weight of the cosmetic or medicinal cream.
